(19) United States
(12) Patent Application Publication
(10) Pub. No.: US 2009/0029432 A1
(43) Pub. Date: Jan. 29, 2009

(54) DRY FRACTIONATION OF CORN

(76) Inventors: Charles Abbas, Champaign, IL (US); Kyle E. Beery, Decatur, IL (US); Thomas P. Binder, Decatur, IL (US); Thomas Gottemoller, Mount Zion, IL (US)

Correspondence Address: BUCHANAN INGERSOLL & ROONEY PC, P.O. BOX 1404, ALEXANDRIA, VA, 22313-1404, US

(21) Appl. No.: 12/179,946

(22) Filed: Jul. 25, 2008

Related U.S. Application Data
(60) Provisional application No. 60/961,875, filed on Jul. 25, 2007.

(51) Int. Cl. C12P 7/10

(52) U.S. Cl. 435/165

(57) ABSTRACT

Novel grain processing methods and the products obtained therefrom are disclosed. Methods may include separation of pericarp fractions, hydrolysis of the pericarp fractions one or more time, and fractionation of the hydrolyzed pericarp fractions. Hydrolyzed pericarp fractions have applications including fermentation media, livestock feed, and fuel feedstocks.

### CLAIM FOR PRIORITY

This application claims priority to pending U.S. Provisional Patent Application No. 60/961,875, filed on Jul. 25, 2007. That application is incorporated by reference as if fully rewritten herein.

### STATEMENT OF FEDERALLY SPONSORED RESEARCH

This invention was made with the assistance of United States Department of Agriculture Grant NRCS 68-3A75-3-140 “Biomass research and Development for the Production of Fuels, Chemicals, and Improved Cattle Feed.” The United States government may have some rights to this invention.

### BACKGROUND OF THE INVENTION

The following includes information that may be useful in understanding the present teachings. It is not an admission that any of the information provided herein is prior art, or material, to the presently described or claimed subject matter, or that any publication or document that is specifically or implicitly referenced is prior art.

1. Field of the Invention

The present teachings relate to, but are not limited to, the field of corn product production. Embodiments relate, for example, to methods for production of ethanol and stillage.

2. Background of the Art

Corn processing methods may be divided into a number of broad groups, including dry grind ethanol, modified dry grind ethanol, corn wet milling and corn dry milling. (Singh, V., et al., “Modified Dry Grind Ethanol Process,” Publication of the Agricultural Engineering Dept. of Univ. of Ill. and Urbana-Champaign, UILU No. 2001-7021, Jul. 18, 2001, incorporated by reference herein). Variation within processes may occur based on the preferences of the miller.

In a typical traditional dry grind ethanol operation, whole corn is ground, mixed with water, treated with alpha-amylase enzyme, and cooked. The resulting “mash” is treated with glucoamylase enzyme to convert it to glucose. The converted mash is fermented and distilled, producing ethanol, distillers dried grains with solubles (DDGS) and carbon dioxide.

Traditional dry grind ethanol operations have a number of disadvantages. For instance, use of the entire kernel in the mash, including the non-starch portions of the kernel, reduces the efficiency of the operation. Furthermore, the non-ethanol byproducts (including DDG) have a relatively low value, and they include a high oil content that is relatively difficult to extract.

In a proposed modified corn dry grind ethanol operation, corn is first cleaned in a dry state to remove cobs and other undesirable components such as metal or stones. The corn may also be wet-cleaned to remove dirt or dust. Following cleaning, the corn is tempered to between about 14% and 22% moisture, typically about 20% moisture. Tempering entails treating the corn with cold water, hot water, and/or steam. This softens the pericarp and causes the germ to become rubbery, which allows those components to be more easily separated from the endosperm.

Following tempering, and while the corn is still moist, the corn is milled to separate portions of the germ, tip cap, and pericarp (bran) from the endosperm, which is customarily used to make grits, meals, and flours. The pericarp and germ proceed through the “through stock” stream, which is dried, cooked, and aspirated. This removes the pericarp, which is dried and used as an animal feedstock. The remaining dried germ, which typically contains about 45% corn oil on a dry basis, is transferred to a separate facility, where the oil is removed through chemical extraction and/or auger press/expeller. The corn residue from the press or extractor is then used as an animal feed.

The non-pericarp, non-germ components of the kernel are ground and converted to mash, as is done in the conventional dry grind ethanol operation. The mash is treated with enzymes to convert it to glucose. The glucose is fermented and distilled, producing ethanol, an animal feed, and carbon dioxide. The animal feed would include yeast cell mass, fermentation by-products, and any other unfermented solids.

Others have attempted to modify the dry grind process to provide higher-value products with varying levels of success. For example, Singh, V., et al., Cereal Chemistry, 73(6): 716-720 (1996), U.S. Pat. No. 6,254,914, to Singh, et al. (“Singh II”), and U.S. Pat. No. 6,899,910, to Singh, et al. (“Singh III”) report removal of germ from soaked corn. The processes include a long tempering phase, and the entirety of each Singh process is performed in the aqueous phase. Singh and Singh II further require addition of substances to the aqueous phase to allow the germ to float and to enable separation by skimming or hydroclone. Singh III reports use of enzymatic hydrolysis of corn starch to increase the density of the aqueous phase and allow separation of the components.

U.S. Pat. No. 6,592,921, to Taylor, et al. reports fractionation of pericarp from corn through addition of ammonia gas to the corn. Taylor, et al. does not teach or suggest the use of water in lieu of ammonia, and it does not teach germ fractionation. U.S. Patent Application No. US20070037267A1, to Lewis, et al. reports corn fractionation to produce a starch-rich stream, though no fractionation is actually described and ethanol is only purportedly produced from residual starch.

U.S. Pat. No. 4,181,748, to Chwalek, et al. reports a dry corn milling process purportedly including use of residual starch in further wet-milling. U.S. Pat. No. 6,962,722, to Dawley, et al., and U.S. Published Application No. US20060057251, to Dawley, et al. report production of a high-protein or mid-protein distillers' dried grain (DDG).

International Patent Publication No. WO2007/015741, to Jansen, et al. and U.S. Pat. No. 6,982,328, to Werpy, et al., report a wet milling process including recovery of starch from fiber. International Patent Publication No. WO 2006/055489, to Beaver, et al., reports separation of corn into a lower starch fraction and a higher starch fraction.

### BRIEF SUMMARY

Generally, various embodiments of the invention provide methods for treatment of grain and grain products to obtain higher value streams from those products in a dry milling operation, exemplified herein by a corn dry milling operation. Embodiments provide new, more cost-effective ways of processing grains prior to fermentation. Typical embodiments provide effective removal of pericarp and separation of germ prior to grinding and saccharifying the endosperm fraction for use in fermentation, resulting in a substantial energy savings. For example removing the non-fermentables from the fermentation could lead to an energy savings of up to 2,800 BTU/liter ethanol produced. Further embodiments include processing of the pericarp fraction by thermochemical hydrolysis and fractionation. This further processing may create product streams with enhanced value and/or utility. In a particularly advantageous embodiment, the hydrolyzed pericarp fraction, which contains solubilized xylose, is added along with the saccharified starch fraction to the fermentation medium thereby increasing the yield of ethanol from the fermentation.

One aspect of the present disclosure is a modified process for ethanol production from a grain in a dry-grind grain processing plant. The process includes separating a pericarp enriched fraction from germ and endosperm enriched fractions of a ground grain, hydrolyzing cellulose and hemicellulose from the separated pericarp fraction to form a xylose enriched soluble fraction, and adding the xylose enriched soluble fraction to a fermentation medium that includes conventional hydrolyzed starch from the endosperm enriched fraction of the grain to produce ethanol. This process of extracting sugars from the otherwise unfermentable pericarp tissue increases the ethanol yield per bushel of grain by as much as 0.3 gallons per bushel.

In a typical practice, hydrolyzing the cellulose and hemicellulose from the pericarp includes thermochemically treating the separated pericarp fraction by exposure to a temperature greater than 25° C. in the presence of at least one of a mineral acid, an organic acid, a mineral base, and an oxidizing agent. Optionally, the hydrolyzing further includes treating the separated pericarp fraction to at least one of a cellulose and a hemicellulose degrading enzyme.

Another aspect involves new methods for separating the pericarp fraction from the endosperm fraction of the grain in a manner that simultaneously produces a better endosperm fraction as a source of the hydrolyzed starch and a better pericarp fraction for solubilization of the cellulose and hemicellulose. In a advantageous embodiment, the pericarp enriched fraction is obtained by aspirating the ground grain by upward flow of gas at a first air pressure in a hopper and harvesting a first fraction of lighter components that are enriched toward an upper portion of the hopper, which are separated from a first fraction of heavier components that are enriched in a lower portion of the hopper. The lighter components are enriched with pericarp while the heavier components are enriched with germ and grain. In a further enhancement, after harvesting the lighter components, the aspirating gas is increased to a second air pressure greater than the first air pressure to separate the first fraction of heavier components into a second lighter fraction enriched with endosperm and a second heavier fraction enriched with germ, which can be harvested separately. The endosperm enriched fraction is liquefied and treated with a starch hydrolyzing agent (saccharification) to provide the hydrolyzed starch which is the primary carbohydrate source for the fermentation medium. This process not only provides an economical way to clean the endosperm, but also provides a more economical way to obtain a relatively clean germ fraction, which can be extracted to obtain oil.

In yet another aspect, the method provides for a way to extract further value from the pericarp fraction. In a conventional dry grind ethanol operation, pericarp containing material from a dry grind ethanol plant is included with the whole grain, or germ extracted grain in the fermentation medium and harvested as distillers dried grains (DDGs) post fermentation. DDGs are typically used as feed supplements for animals. However, pericarp tissue that has been hydrolyzed to yield soluble xylose containing material which is then used for the fermentation has less value as a feed. In certain embodiments of the present teaching the non-soluble pericarp material obtained after hydrolysis and removal of the xylose containing soluble fraction, is further treated by at least one process selected from pyrolysis and hydrothermal upgrading. The product of such pyrolysis or hydrothermal upgrading is a crude oil like substance (i.e., a “biocrude”), that can be used as starting material for further fractionation to make a fuel or fuel additive or used directly as a crude fuel material.

One exemplary embodiment of a process disclosed herein is, in a dry grind grain processing plant, tempering a dry weight of grain by adding about 10% wt of water per dry weight of grain and heating to a temperature of about 20-40° C. for a period sufficient to swell a germ component of the grain; grinding the tempered grain; separating the ground grain by aspiration to obtain a first fraction enriched with pericarp and a first amount of starch fines and a second fraction enriched with endosperm and germ; separating the first fraction by sizing to form a pericarp enriched fraction and a fines enriched fraction; separating the second fraction into an endosperm enriched fraction and a germ enriched fraction; adding water and a hydrolytic agent to the pericarp enriched fraction to form a first mixture and heating the first mixture for a time and temperature sufficient to hydrolyze at least 45% of the fiber in the pericarp into a soluble sugar fraction containing xylose. The soluble sugar fraction containing xylose is then used to supplement the fermentation medium.

The methods provided herein increase the yield of ethanol per bushel of corn to between about 2.7 to about 3.0 gallons per bushel in comparison to a yield of about 2.4 to 2.7 gallons by conventional dry grind fermentation.

### BRIEF DESCRIPTION OF THE FIGURES

FIG. 1 depicts a conventional dry mill ethanol production process. (Singh, et al.).

FIG. 2 depicts a modified dry mill ethanol production process. (Singh, et al.).

FIG. 3 depicts a flow chart of a dry mill ethanol production process according to one embodiment of the invention.

FIG. 4 depicts a flow diagram of a modified dry milling process.

FIG. 5 depicts a flow diagram of a modified dry milling process according to an embodiment of the invention.

FIG. 6 depicts a dry mill ethanol production process of a further embodiment of the invention, as reported in Example 3, below.

FIG. 7 depicts a flow chart of an embodiment of the invention.

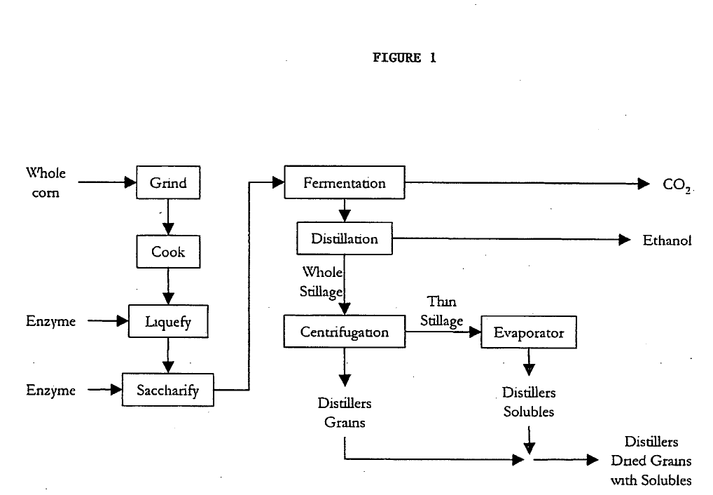

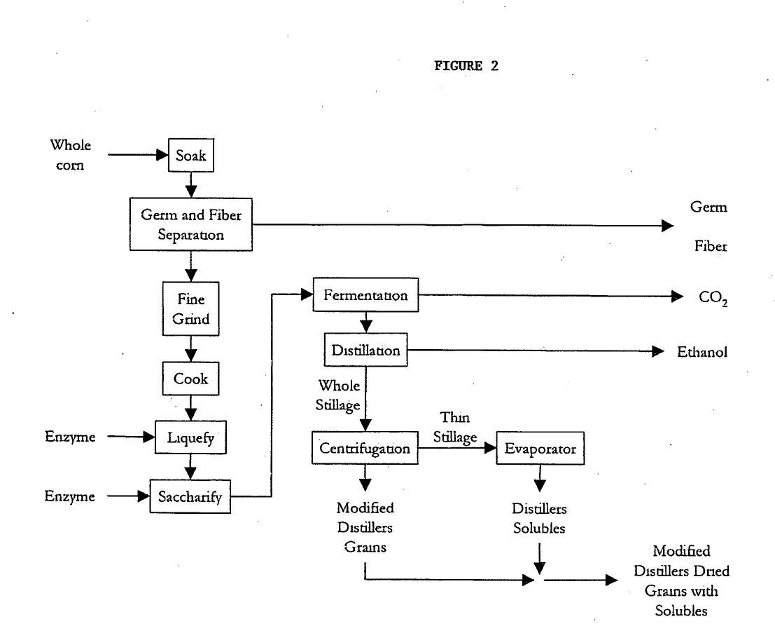

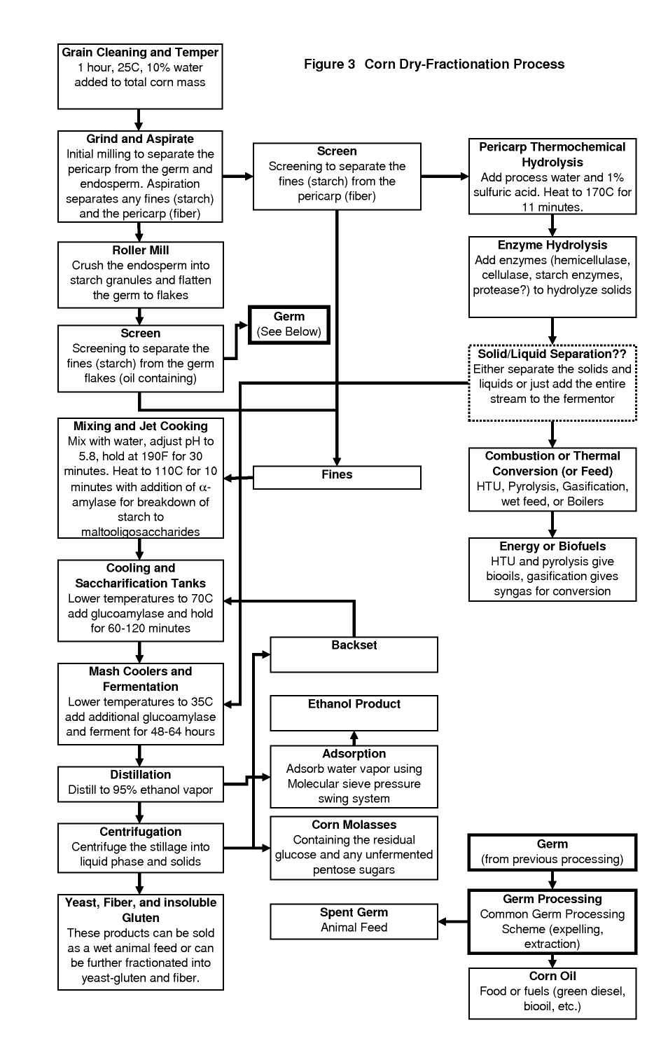

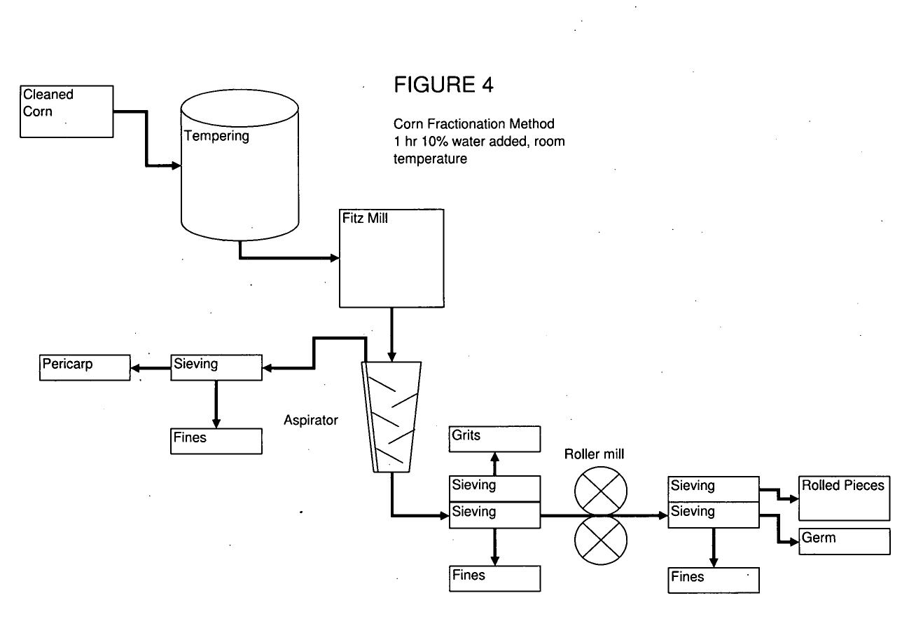

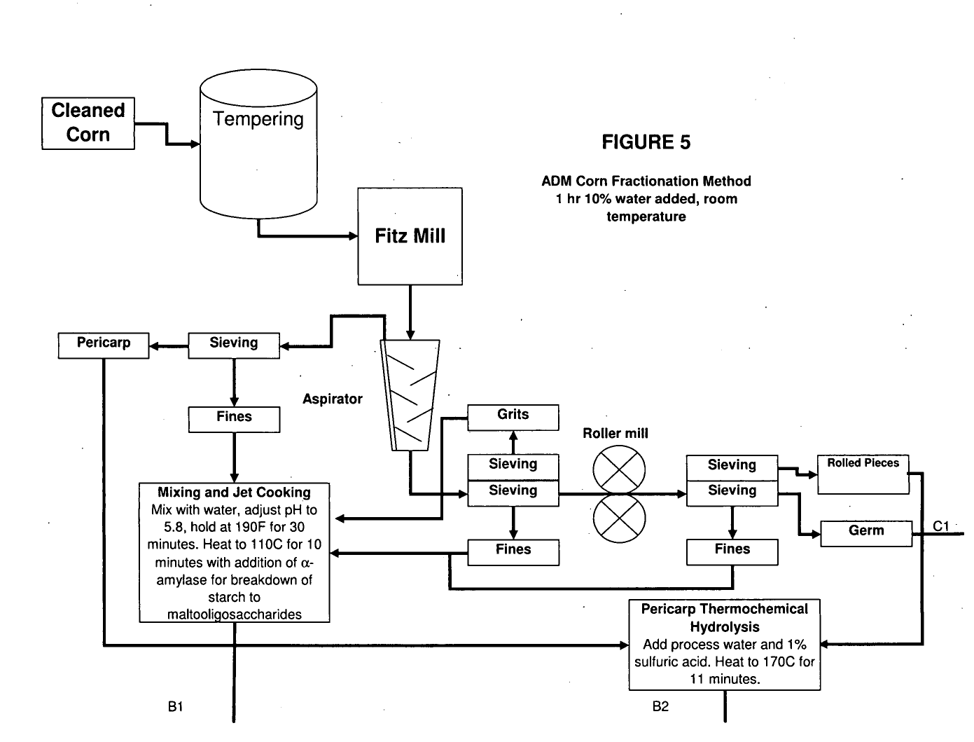

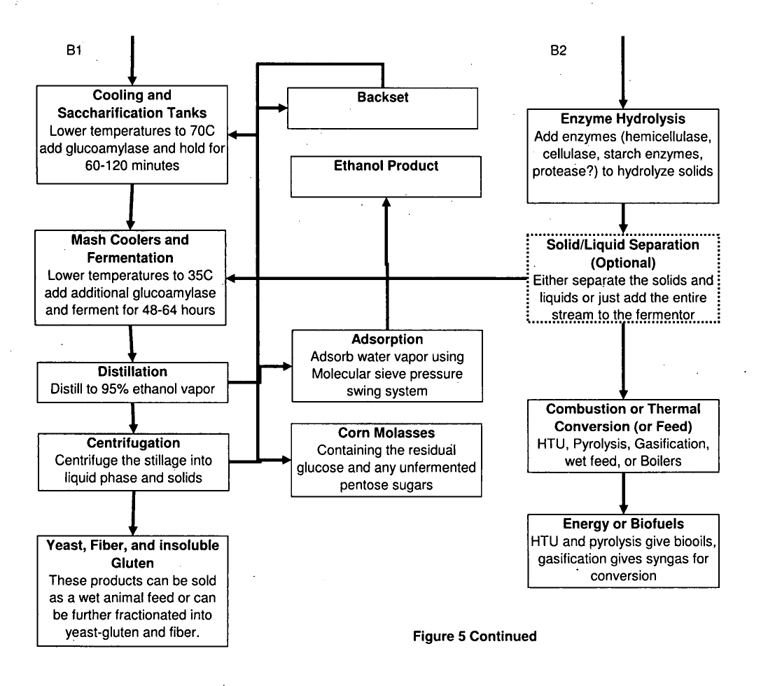

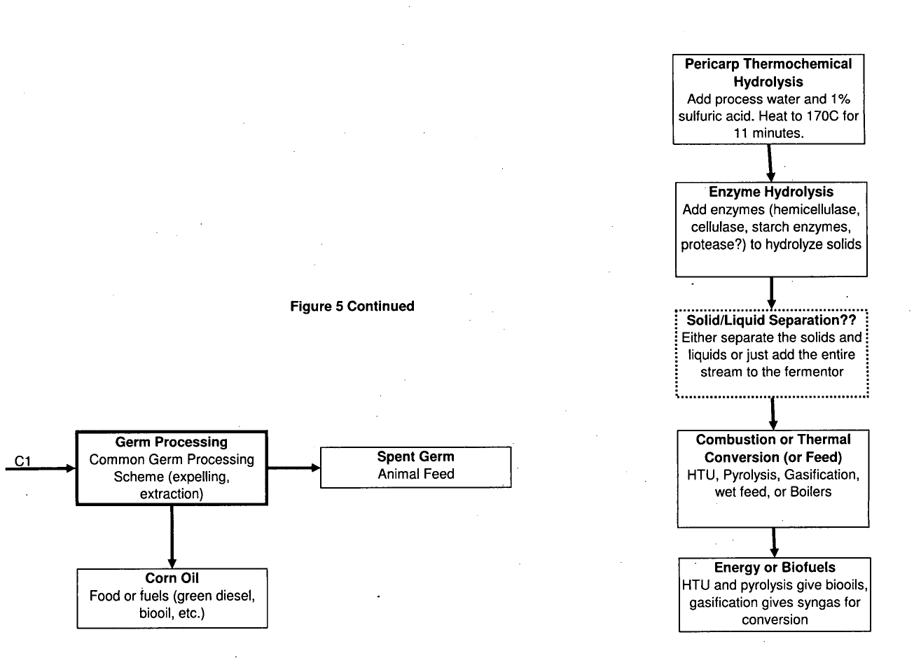

### DETAILED DESCRIPTION OF THE INVENTION

The present teaching describes several different features and aspects of the invention with reference to various exemplary embodiments. It is understood, however, that the invention embraces numerous alternative embodiments, which may be accomplished by combining any of the different features and aspects described herein in any combination that one of ordinary skill in the art would find useful.

Processing methods as described herein may offer many advantages over the prior art. Of course, the scope of the invention is defined by the claims, and whether an embodiment is within that scope should not be limited by whether the method provides one or more of these advantages. Processing methods may require lower energy input, lower capital costs, and lower processing costs than other methods known in the art. Minimal water input may be required. Processing according to embodiments presented herein may be particularly advantageous for ethanol processing; for example, processes provided herein may increase ethanol produced per bushel of corn from about 2.7 gallons per bushel to between 2.9 and 3.0 gallons per bushel. Hydrolyzed pericarp fiber may be used to provide between about 7,000 and 17,000 BTU/bushel of corn if returned to the boilers used to provide energy for the aspiration or related processes. Hydrolyzed pericarp produced by embodiments of the invention may also be used as fuel or a fuel precursor for a variety of applications, including but not limited to biodiesel, biooil, and syngas.

A. Corn Processing

Discussion of the methods and compositions taught herein will be made using corn as an exemplary grain for the practice of the invention. Those skilled in the art will, with the benefit of this disclosure, recognize that although methods and compositions are described with respect to corn (maize), the methods and compositions may be beneficial when practiced with other grains and grain-like substances. Grains or grain-like raw materials for use with the teachings herein may include, for example, but are not limited to, wheat, millet, barley, sorghum, triticale, rice, corn, amaranth, buckwheat, rye, oats, and quinoa. Corn is preferred. No particular strain of corn is required.

Embodiments provide methods for processing corn in a dry milling or modified dry milling process. One embodiment for providing ethanol and stillage includes the steps of tempering whole corn to soften and loosen the germ and pericarp. Prior to tempering, the corn may be cleaned to remove rocks, dirt, or other undesired foreign matter. For tempering, water may be added at varying amounts by weight. Addition of 10% water by weight of the corn is preferred, though in other embodiments the amount of added water is between about 5% and about 15% by weight of the corn stream, and in other embodiments, the amount of added water is between 0 and 20%.

Following addition of water, the corn is held at 25° C., or between 20 and 30° C., and continuously mixed for a period of time to complete the tempering. The temperature of the corn/water mixture is between 15 and 35° C., preferably about 25° C. If heating the mixture is necessary, it is preferably done using steam. The time of mixing is 0 to 120 minutes, preferably 15-60 minutes. Mixing may be conducted by any mixer, including but not limited to ribbon mixer, auger, or blender.

Following tempering, the corn is ground or milled. In a separate embodiment, the corn can be ground or milled without tempering, however, tempering improves the separation of pericarp material as described herein after. Milling may be done by any method. Preferred methods include milling by Fitz mill or by Beall degerminator or Satake degerminator. Milling and grinding produce a ground corn stream, which is a heterogeneous mixture including germ, endosperm, pericarp, and fines. The fines are primarily starch, and the pericarp is primarily fibrous material.

The ground corn stream is then separated to remove the oil-containing germ and the starch/gluten endosperm from the pericarp and fines. In a preferred embodiment, the germ and endosperm are separated from the pericarp and fines by aspiration. The aspiration can be completed in a Kice gas aspirator using a differential pressure of 0.5-1.0 inches of water. The heavy fraction contains the germ and endosperm grits, while the light fraction is a pericarp enriched fraction containing the pericarp and fines. Residence time in the aspirator is a function of the height and width of the aspirator. Parameters such as flow rate and residence time will depend on the height, width, and other physical dimensions of the aspirator.

Following separation of the pericarp and fines from the germ and endosperm, the pericarp and fines are separated to provide a pericarp fraction and a fines fraction. In a typical embodiment the separation is performed by sieving. This can be accomplished in a Sweco shaking screener with a U.S. standard sieve size of 12. The −12 (through or under 12) is part of the endosperm or starch fraction and the +12 (over 12) is the pericarp fraction. Typically, the pericarp fraction at this stage still contains about 15%-30% attached starch on a weight basis. After separation, the pericarp may be treated and used a variety of ways, as set forth in more detail in Section B, below.

Once separated from the pericarp and fines, the remaining germ and endosperm can be prepared for ethanol fermentation. There are several possible methods for this. One method is to aspirate the heavys (throughs) from the first aspiration operation at a higher differential pressure. For example, at a differential pressure of 2.5 to 3.5 inches of water, a heavy fraction could be separated from a light fraction. The heavy fraction is enriched with endosperm and the light fraction contains a mixture of germ and endosperm. After aspiration, the heavy fraction can be added directly to the starch fraction, and the light fraction can be sieved using a U.S. standard sieve size of 6. After sieving, the +6 fraction contains primarily germ tissue and the −6 fraction contains primarily endosperm tissue.

In all cases, the endosperm fractions or high starch fractions can be combined into a single fraction for fermentation to ethanol.

In another embodiment, the heavy fraction from the first aspiration, containing the germ and endosperm grits fractions, is sieved to separate out the fines and large grits from the intermediate fraction. This may be accomplished utilizing U.S. standard sieve sizes of 20 and 10 in a Sweco shaking screen. The +20-10 (intermediate) fraction can then be milled, fracturing the endosperm into starch granules (fines) and forming the germ fraction into thin flakes. Milling may be done, for example, in a Ferrell-Ross flaking roller mill. The fines, other large endosperm pieces, and germ flakes are separated by sieving, aspiration, or other suitable method. The fines may then be combined with the fines and other high-starch composition fractions that were previously separated from the pericarp and germ.

The separated germ flakes or whole germs may be processed in a number of ways. For example, they may be pressed in an expeller, or they may be subjected to an extraction. They may pressed by an expeller and then subjected to an extraction. These processes will provide high-value corn oil.

In one embodiment, the fines and other high-starch composition fractions are processed with a moisture source and by heat and enzymatic activity to provide a fermentation medium suitable for ethanol fermentation with Saccharomyces cerevisiae or another ethanol-producing microorganism. A fermentation medium is prepared, for example, by mixing the endosperm fraction with a volume of water having a weight of two to three times the weight of the fines. This produces a slurry. Water may be obtained from any source, but in typical embodiments it is fermentation backset, condensed evaporator water, or corn steep liquor. The temperature of the slurry is maintained at between 70 and 90° C. The pH of the slurry is adjusted to between to 5.2 and 6.0, typically 5.8. If the initial pH of the slurry is more acidic than the target pH, the pH may be adjusted, for example, by addition of sodium hydroxide or another base; if the slurry is initially too basic, its pH may be adjusted, for example, with addition of sulfuric acid or another acid.

After the target slurry pH has been reached, the slurry is raised to between 82.2° C. and 93.3° C., preferably 87.8° C., and then held at the selected temperature for about 30 minutes. The slurry is then liquefied with addition of α-amylase enzyme and heated to 105-110° C. for 5 to 10 minutes. In some embodiments, the slurry is heated to between 105 and 110° C., preferably about 110° C. for between 5 and 15 minutes, preferably 10 minutes. The addition of the enzyme combined with heating causes breakdown of starch to maltooligosaccharides. The resulting slurry is then further treated by reduction of the temperature to between 60 and 75° C., preferably 70° C., addition of glucoamylase, adjustment of pH to about 4.5, and maintenance of the selected temperature for between 1 and 48 hours to saccharify the maltooligosaccharides to glucose monosaccharide. In a typical embodiment the resulting slurry is used as a fermentation media for ethanol production with a fermentative microorganism.

The separated pericarp can be utilized as an animal feed. In an alternative embodiment, treated pericarp as described below is added to the media to increase the available saccharides for fermentation. Fermentation will typically include lowering the temperature of the fermentation mixture to between about 30 and 40° C., typically 35° C., with addition of glucoamylase and at least one fermentative microorganism. A fermentative microorganism may be, for example, a yeast, bacteria, or fungus. Glucoamylase may be added at the does recommended by the manufacturer; typical dosages are 0.22 units of glucoamylase/gram of starch. Other enzymes may also be added including hemicellulases, proteases, cellulases, and feruloyl esterases to break down other soluble oligosaccharides to monosaccharides or to assist in the enzymatic conversion of the oligosaccharides.

Fermentative ethanol is distilled, resulting in an ethanol stream and a remainder stream of yeast, gluten, water, non-starch fine fiber, and other non-soluble solids. The spent fermentation broth may be separated into a liquid fraction and a solids fraction, or the liquids may be evaporated to create or supplement a high solids animal feed. In alternative embodiments the separation is performed by a press, centrifuge, filter, or evaporator. The separated solids may be used, for example, as a wet animal feed. The separated liquids may be used, for example, as process water or as a liquid animal feed.

B. Pericarp Processing

Embodiments of the invention provide various treatments and uses for pericarp. Typically, pericarp and fines are first separated from the tempered, milled corn, and the pericarp is subsequently separated from the fines. In one embodiment, the pericarp undergoes no further processing and is sold. For example, the pericarp may be sold as an animal feed.

In a further embodiment, the pericarp is hydrolyzed a first time using heat with the addition of acid. In this thermochemical hydrolysis, the pericarp is mixed with water and acid, then heated. Preferably water is added until the pericarp/water mixture is at least 40% by weight, more preferably 45% by weight. The pericarp/water ratio may be 60% pericarp to 40% pericarp. Sulfuric acid at a 1% concentration is the preferred acid for addition, but other acids that may be used include hydrochloric acid, nitric acid, peracetic acid, acetic acid, lactic acid, phosphoric acid, succinic acid, citric acid, and maleic acid. Sufficient acid is added as needed for hydrolysis. The mixture is heated to between 145 and 200 C, preferably 170° C., for between 0.1 and 60 minutes, preferably 11 minutes. This thermochemical hydrolysis not only hydrolyzes cellulose and hemicellulose to produce a solubilized fraction enriched with oligosaccharides and monosaccharides such as xylose, (a slurry), but also hydrolyzes most of the starch that is still attached to the pericarp tissue into oligosaccharides and glucose.

Following the initial hydrolysis, the hydrolyzed pericarp slurry may be washed and pressed to provide a liquid solubilized fraction and a solid fraction. In the alternative, the slurry may be maintained as a mixture.

1. Separation of the Hydrolyzed Pericarp Slurry

If the hydrolyzed pericarp slurry is separated, the liquid fraction (containing hemicellulose and starch oligosaccharides) may be further hydrolyzed a second time to convert the oligosaccharides into monosaccharides, particularly xylose and arabinose from the solubilized hemicellulose, but also glucose from the solubilized cellulose and starch. In an alternative embodiment, the liquid fraction is used to produce chemicals. The chemical intermediates that may be produced from the oligosaccharides and monosaccharides include dehydrosugars, furans, levulinic acid, and formic acid. These chemicals may be used to produce polymers, fuel oxygenates, solvents, and many other chemicals.

If the liquid fraction of the hydrolyzed pericarp slurry is hydrolyzed a second time, the second hydrolysis is conducted with the addition of enzymes or other chemicals to facilitate the conversion of oligosaccharides into monosaccharides. For example, the treatment conditions may be 1% by weight sulfuric acid added to the hydrolysate and heated in a reactor to 121° C., 0.110 MPa for 30 minutes. For the enzymatic hydrolysis, the pH may be adjusted to pH 5-6 and hemicellulases, cellulases, feruloyl esterases, and proteases may be added to the hydrolysate and the mixture may be held at 60° C. for 2 to 48 hours. Amylase and glucoamylase may also be included to further solubilize starch derived oligosaccharides. The twice-hydrolyzed liquid fraction may be used as a stand-alone fermentation media, or it may be used to supplement a separate fermentation media. This includes, for example, but is not limited to, an endosperm-derived media as described in Section A herein.

The solids fraction of the hydrolyzed pericarp has a variety of uses. For example, it may be used as an animal feed, as a boiler feed, or as a feedstock for pyrolysis, biooil, gasification, or hydrothermal upgrading. Pyrolysis and hydrothermal upgrading each involve exposing the solids to increased temperature and pressure for various times to liquefy the material, or at least a portion of the material. These liquefied materials can then be hyrdro-processed by the addition of hydrogen gas over a catalyst to produce biooils. Tables 1 and 2 show the typical conditions for pyrolysis and hydrothermal upgrading (liquefaction).

TABLE 1
Comparison of Liquefaction Process Operations

| | | hydrothermal |
|---|---|---|
| | Pyrolysis | liquefaction* |
| operating temperature | 450-500° C. | 350° C. |
| operating pressure | 1 atm | 200 atm |
| residence time | <1 sec | 19 min |
| oil product yield | 70-75% wet bio-oil | 50% dry oil |
| oil product quality | | |
| heating value (HHV, | 6886 Btu/lb | 14200 Btu/lb |
| Btu/lb) | | |
| oxygen content | 40% | 15% |
| water content | 25% | 5% |
| viscosity@60° C. | low (10 cps) | high (17,000 cps) |
| thermal stability | No | Yes |
| distillable | light ends and water | half to two-thirds |
| | only | |

TABLE 2
Further Hydroprocessing of Pyrolysis and Hydrothermal
Liquefaction materials

| | | hydrothermal |
|---|---|---|
| | Pyrolysis | liquefaction |
| operating | low temp required | typical (350-450° C.) |
| temperature | (250° C.) before | |
| | finishing step | |
| | (400° C.) | |
| catalysts | Pd, Ru, Ni, CoMo, NiMo | typical (NiMo or CoMo) |
| liquid hourly space | 0.1 | 0.19 |
| velocity | | |
| product gasoline | 0.3 liter/liter feed | 0.8 liter/liter feed |
| yield | | |
| oil product quality | gasoline and diesel range | gasoline and diesel range |
| | hydrocarbons | hydrocarbons |
| oxygen content | 1.3 | 0.1 |
| H/C ratio | 1.6 | 1.5 |
| water content | 500 ppm | very low |

2. Utilization of Hydrolyzed Pericarp in a Mixture

If the hydrolyzed pericarp is not separated, then the combined liquid and solid fractions may be treated with enzymes to hydrolyze the mixture a second time to partially or completely hydrolyze the solids to oligosaccharides and monosaccharides and partially or completely convert soluble oligosaccharides to monosaccharides. Suitable enzymes and accessory enzymes include, for example, but are not limited to proteases, cellulases, hemicellulases, feruloyl esterases, and starch-degrading enzymes. The enzyme hydrolysis may occur, for example, at 60° C. for 2 to 72 hours. The twice-hydrolyzed slurry may then be used as a fermentation media for ethanol or other chemicals. It may also be used to supplement a fermentation media as described in Section A herein. In the alternative, the twice-hydrolyzed slurry may be used to produce animal feed, boiler feed. After fermentation, any residual carbohydrates in the monosaccharide, oligosaccharide or polysaccharide form may be used as an animal feed or for pyrolysis, biooil production, gasification, or hydrothermal treatment.

### EXAMPLES

The examples below are only representative of some aspects of the invention. These examples should not be interpreted as limiting the invention in any way not explicitly stated in the claims.

### Example 1

Corn milling tests have been conducted on dry fractionation of corn kernels at ADM. This run consisted of placing 5 kg of corn kernels in a rotating sealed vessel and adding 10% water. The vessel was rotated for 1 hour at room temperature and then the kernels were removed. The tempered corn kernels were roughly ground through a Fitz Comminutor fitted with a ¼″ screen; followed by aspiration through a Kice aspirator with a 1 inch of water differential; the “overs” and “throughs” from the aspirator were sieved in a Sweco shaking screener at 6, 12, and 20 mesh sizes. After sieving, the intermediate particles (−6/+20) from the “throughs” were roller milled twice at a gap setting of 1.1 on the Ferrell-Ross Flaking roller mill and then sieved in a Sweco shaking screener at 6 and 12 mesh sizes. The fines (20 mesh or below, −20) were combined prior to analysis. This produced 6 fractions as shown in Table 1 below. With the exception of the pericarp and the germ, the remaining listed components form the endosperm.

The results show that the fines are highly enriched in starch as compared to the native kernels, and depleted of NDF (neutral detergent fiber, equivalent to hemicellulose, cellulose and lignin), fat and protein. This fines fraction is the largest fraction at 35.6%. Other samples enriched in starch include the Grits (33.6% of the yield) and Rolled Fines (10.1% of the yield). These fractions are also enriched in protein and depleted in NDF. The Rolled Pieces and the Grits fractions are compositionally the most similar to the overall corn kernel composition.

The pericarp fraction can be hydrolyzed by mixing the pericarp with water (or optionally, backset, corn steep liquor or other process water) until the moisture level is 40-60%. The pericarp slurry can then be placed into a pressurized reactor and 1 wt/wt % sulfuric acid added. The reactor can be heated by direct or indirect steam heating, or by electrical heating. The reactor is heated to 120 to 200° C. for 1 to 120 minutes, preferably to 150-175° C. for 11 to 30 minutes. The pressure in the reactor will be from approximately 2 bars at 120° C. to approximately 16 bars at 200° C.

After the initial hydrolysis has occurred, the hydrolyzed pericarp slurry is optionally mixed with an additional water source, which could include corn steep liquor, backset, water or process water. The slurry is cooled to approximately 60° C. and enzymes such as hemicellulases, cellulases, proteases, and feruloyl esterases are added to hydrolyze the hydrolyzed solids and soluble oligosaccharides. The slurry can then be added to the fermentation media prepared from the starch enriched fraction.

TABLE 1
10% Moisture Tempered Corn - Fractions Compositions (%)

| | Yield | Protein | Ash | Fat | NDF* | Starch |
|---|---|---|---|---|---|---|
| Corn Kernels | | 7.42 | 1.3 | 3.94 | 10.8 | 71.38 |
| Fines | 35.60 | 6.16 | 0.60 | 2.18 | 2.90 | 87.15 |
| Grits | 33.60 | 9.39 | 1.10 | 4.21 | 5.06 | 76.95 |
| Rolled Fines | 10.10 | 7.97 | 0.64 | 2.74 | 3.45 | 83.85 |
| Germ | 2.70 | 16.50 | 6.29 | 19.47 | 17.45 | 33.59 |
| Pericarp | 10.00 | 8.78 | 1.68 | 3.97 | 43.30 | 36.57 |
| Rolled Pieces | 8.00 | 12.10 | 2.97 | 8.54 | 9.02 | 64.07 |

The resulting products from this processing yield products as shown in Table 2, below. Table 2 compares the expected product yield of untreated corn with corn treated as described in this example. Treated corn increased ethanol production (2.976 gal/bu, compared to 2.700 gal/bu), increased yield of biodiesel, and increased boiler energy production. Production of animal feed is decreased by about 9 pounds/bu, but the other beneficial products and uses have a greater value (particularly for supplying energy) and are therefore more desirable. The animal feed that is produced may be used for many species, including ruminants.

TABLE 2
Product Yields

| | Ethanol, | | Boiler Energy, | Animal |
|---|---|---|---|---|
| | gal/bu | | Btu/bu (7300 Btu/lb) | Feed, lb/bu |
| | | Biodiesel, | | |
| | | lb/bu | | |
| Corn | 2.700 | | | 17 |
| | | Biodiesel, | | |
| | | kg/bu | | |
| Endosperm | 2.812 | | | 7.18 |
| Germ | | 0.227 | | 0.77 |
| Pericarp/Hull | 0.264 | | 17,520 | |

### Example 2

Example 2 describes the separation and utilization of the liquids and solids from the hydrolyzed pericarp stream. After the sulfuric acid hydrolysis of the pericarp fraction as described in Example 1, the hydrolyzed pericarp slurry is processed using a screw press or centrifuge to separate the solids and liquid. For example a Vincent screw press model CP-4 could be utilized to separate the solids and liquids. The solids can be washed in the press or centrifuge also to remove more soluble oligosaccharides and monosaccharides from the remaining hydrolyzed solids.

The separated liquid from the hydrolyzed pericarp slurry can be hydrolyzed again with an additional acid hydrolysis or with an enzyme hydrolysis. This will break down the soluble starch and hemicellulose oligosaccharides to monosaccharides that can be fermented to ethanol or other chemicals. The conditions for the acid hydrolysis are 121° C. (approximately 2 bar) for 30 minutes. The conditions for the enzyme hydrolysis are 60° C. for 1 hour using starch hydrolyzing enzymes, hemicellulases, and feruloyl esterases.

The separated solids may be optionally dried and pelletized and sold as an animal feed. Or optionally, the separated solids may be heated in a reactor at various conditions to either pyrolyze, thermochemically treat, hydrothermally treat, or gasify the solids. These conditions will produce oils and gases that can be utilized as fuels and chemicals.

### Example 3

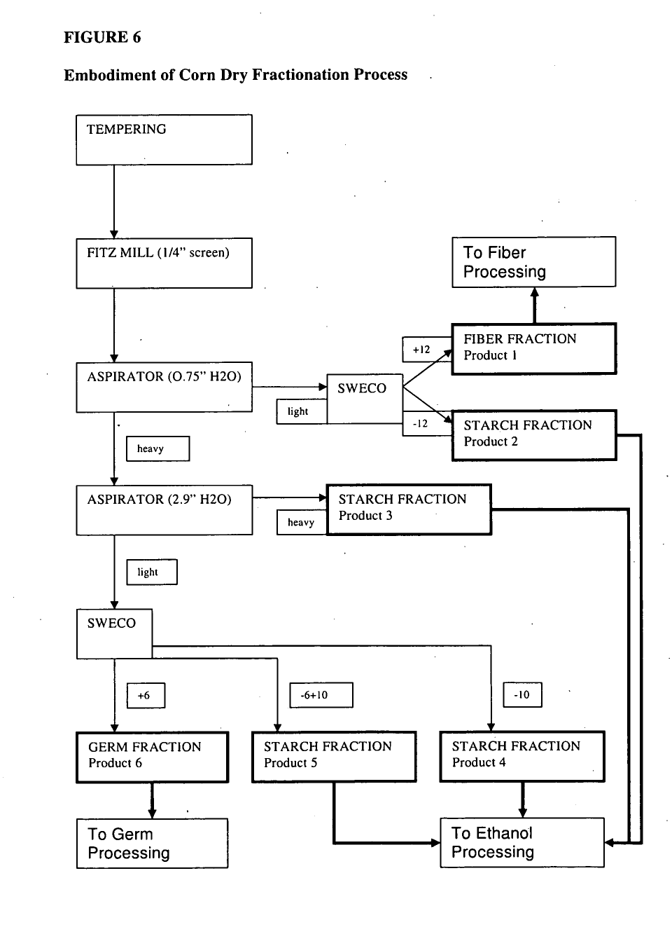

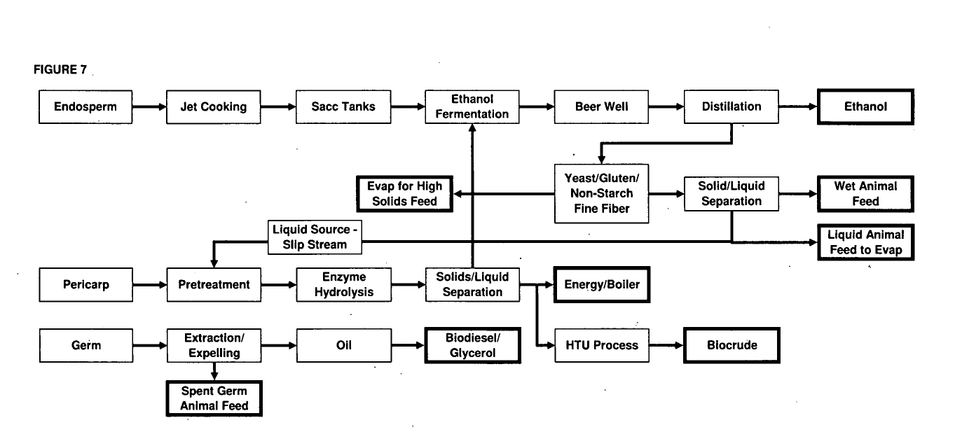

Example 3 reports an additional embodiment of the invention, the flow chart for which may be viewed as FIG. 6. The corn was tempered for 15 to 60 minutes and milled as described in Example 1 above. Then the ground corn was aspirated in a Kice aspirator at a differential pressure of 0.75 inches of water. The light fraction or overs were sieved in a Sweco shaking screener using a U.S. standard sieve size of 12. The +12 fraction consists of the pericarp fraction (Fiber Fraction, Product 1, in FIG. 6) and the −12 fraction consists of an endosperm fraction (Starch Fraction, Product 2, in FIG. 6).

The heavys or throughs were then aspirated again at 2.9 inches of water and the heavy fraction was an endosperm fraction (Starch Fraction, Product 3, in FIG. 6). The lights were sieved in a Sweco sieving screen using U.S. standard sieve sizes of 6 and 10. The +6 fraction was the germ fraction (Germ Fraction, Product 6, in FIG. 6), and the other two fractions were endosperm fractions (Starch Fraction, Product 4, and Starch Fraction, Product 5, both in FIG. 6).

Product analysis for this embodiment is shown in Table 3.

TABLE 3
Product Analysis for Example 3

| | | % of Total Product |
|---|---|---|
| Run Number | Product Number | (as-is) |
| 1 | 1 | 11.3 |
| | 2 | 27.3 |
| | 3 | 11.5 |
| | 4 | 22.0 |
| | 5 | 25.1 |
| | 6 | 2.8 |
| 2 | 1 | 10.6 |
| | 2 | 26.6 |
| | 3 | 10.8 |
| | 4 | 23.4 |
| | 5 | 25.6 |
| | 6 | 3.0 |
| 3 | 1 | 11.6 |
| | 2 | 26.1 |
| | 3 | 11.6 |
| | 4 | 22.8 |
| | 5 | 24.8 |
| | 6 | 3.3 |
| 4 | 1 | 9.5 |
| | 2 | 27.9 |
| | 3 | 11.5 |
| | 4 | 23.6 |
| | 5 | 24.6 |
| | 6 | 3.0 |

Patents, patent applications, publications, scientific articles, books, web sites, and other documents and materials referenced or mentioned herein are indicative of the levels of skill of those skilled in the art to which the inventions pertain, as of the date each publication was written, and all are incorporated by reference as if fully rewritten herein. Inclusion of a document in this specification is not an admission that the document represents prior invention or is prior art for any purpose.

The terms and expressions employed herein have been used as terms of description and not of limitation, and there is no intention in the use of such terms and expressions, or any portions thereof, to exclude any equivalents now know or later developed, whether or not such equivalents are set forth or shown or described herein or whether or not such equivalents are viewed as predictable, but it is recognized that various modifications are within the scope of the invention claimed, whether or not those claims issued with or without alteration or amendment for any reason. Thus, it shall be understood that, although the present invention has been specifically disclosed by preferred embodiments and optional features, modifications and variations of the inventions embodied therein or herein disclosed can be resorted to by those skilled in the art, and such modifications and variations are considered to be within the scope of the inventions disclosed and claimed herein.

Specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. Where examples are given, the description shall be construed to include but not to be limited to only those examples.

It will be readily apparent to one skilled in the art that varying substitutions and modifications may be made to the invention disclosed herein without departing from the scope and spirit of the invention, and from the description of the inventions, including those illustratively set forth herein, it is manifest that various modifications and equivalents can be used to implement the concepts of the present invention without departing from its scope. A person of ordinary skill in the art will recognize that changes can be made in form and detail without departing from the spirit and the scope of the invention. The described embodiments are to be considered in all respects as illustrative and not restrictive. Thus, for example, additional embodiments are within the scope of the invention and within the following claims.

We claim:

1. A modified process for ethanol production from a grain, comprising:
in a dry-grind grain processing plant, separating a pericarp enriched fraction from germ and endosperm enriched fractions of a ground grain;
hydrolyzing cellulose and hemicellulose from the separated pericarp fraction to form a xylose enriched soluble fraction;
adding the xylose enriched soluble fraction to a fermentation medium comprising hydrolyzed starch from the endosperm enriched fraction of the grain; and
fermenting the medium to produce ethanol.

2. The process of claim 1 wherein the hydrolyzing includes thermochemically treating the separated pericarp fraction by exposure to a temperature greater than 25° C. in the presence of at least one of a mineral acid, an organic acid, a mineral base, and an oxidizing agent.

3. The process of claim 2 wherein the hydrolyzing further includes treating the separated pericarp fraction to at least one of a cellulose and a hemicellulose degrading enzyme.

4. The process of claim 1 wherein the pericarp enriched fraction is obtained by aspirating the ground grain by upward flow of gas at a first air pressure in a hopper and harvesting a first fraction of lighter components that are enriched toward an upper portion of the hopper being separated from to a first fraction of heavier components that are enriched in a lower portion of the hopper.

5. The process of claim 4 wherein after harvesting the lighter components, the aspirating gas is increased to a second air pressure greater than the first air pressure to separate the first fraction of heavier components into a second lighter fraction enriched with endosperm and a second heavier fraction enriched with germ, and; harvesting the germ enriched fraction and the endosperm enriched fraction.

6. The method of claim 5 wherein the endosperm enriched fraction is liquefied and treated with a starch hydrolyzing agent to provide the hydrolyzed starch for the fermentation medium.

7. The method of claim 5 wherein oil is extracted from the germ enriched fraction.

8. The method of claim 1 wherein the xylose enriched solubilized fraction is separated from non soluble pericarp material and the non-soluble pericarp material is further treated to at least one process selected from pyrolysis and hydrothermal upgrading.

9. The process of claim 1 wherein separating the pericarp enriched fraction from the germ and endosperm enriched fractions includes moistening the dry grain by adding about 10% wt of water per wt of dry grain and tempering the moistened grain by incubating at 20-40° C. for a period of at least 15 minutes.

10. A modified process for ethanol production, comprising:
in a dry grind grain processing plant, tempering a dry weight of grain by adding about 10% wt of water per dry weight of grain and heating to a temperature of about 20-40° C. for a period sufficient to swell a germ component of the grain;
grinding the tempered grain;
separating the ground grain by aspiration to obtain a first fraction enriched with pericarp and a first amount of starch fines and a second fraction enriched with endosperm and germ;
separating the first fraction by sizing to form a pericarp enriched fraction and a fines enriched fraction;
separating the second fraction into an endosperm enriched fraction and a germ enriched fraction;
adding water and a hydrolytic agent to the pericarp enriched fraction to form a first mixture and heating the first mixture for a time and temperature sufficient to hydrolyze at least 45% of the fiber in the pericarp into a soluble sugar fraction containing xylose.

11. A method for fractionation of a corn stream into endosperm, germ, and pericarp fractions, comprising:
a. tempering a corn stream;
b. aspirating said tempered corn stream at a first differential pressure, thereby producing at least a first light fraction and a first heavy fraction;
c. separating said first light fraction into at least a pericarp fraction and a first endosperm fraction;
d. aspirating said first heavy fraction at a second differential pressure, thereby producing at least a second endosperm fraction and a second light fraction; and
e. separating said second light fraction into at least a third endosperm fraction and a germ fraction.

12. The method of claim 11, wherein said second differential pressure is greater than said first differential pressure.

13. A method for fermentative production of ethanol from corn, comprising:
a. fractionating corn according to the method of claim 11;
b. providing a fermentation media comprising said pericarp fraction, said first endosperm fraction, said second endosperm fraction, and said third endosperm fraction; and
c. fermenting said fermentation media, thereby producing ethanol.

14. The method of claim 13, wherein the yield of ethanol per bushel of corn is between about 2.7 to about 3.0 gallons per bushel of corn.

15. A method for providing a hydrolyzed pericarp fuel, comprising:
a. fractionating corn according to the method of claim 11;
b. hydrolyzing said pericarp fraction, thereby producing a hydrolyzed pericarp fuel.

16. The method of claim 15, further comprising separating said hydrolyzed pericarp into a liquid stream and a solid fraction, wherein said solid fraction is a hydrolyzed pericarp fuel.

17. A method for fractionating corn into germ, endosperm, and pericarp fractions, comprising:
a. tempering a corn stream;
b. grinding the tempered corn stream to provide a mixture comprising pericarp, fines, germ, and endosperm;
c. aspirating said mixture to produce a fraction comprising pericarp and starch fines and a fraction comprising germ and endosperm;
d. separating said fraction comprising pericarp and starch fines to provide a pericarp fraction and a first starch fines fraction;
e. separating said fraction comprising germ and endosperm to provide a second starch fines fraction and a germ fraction; and
f. combining said first starch fines fraction and said second starch fines fraction to produce an endosperm fraction.

18. The method of claim 17, wherein said fraction comprising germ and endosperm is separated by at least one method selected from the group consisting of aspiration and sieving.

19. A method for providing ethanol and stillage from a whole corn stream, comprising:
a. tempering a whole corn stream by adding 10% water by weight and holding for 15 minutes to one hour at about 25° C., producing a tempered corn stream;
b. grinding said tempered corn stream to produce a ground corn stream mixture comprising pericarp, fines, germ and endosperm;
c. aspirating said ground corn stream mixture to produce a pericarp and fines fraction and an endosperm and germ fraction;
d. screening said pericarp and fines fraction to produce a pericarp fraction and a first fines fraction;
e. hydrolyzing said pericarp fraction by adding water to increase the total weight to at least 1.67 times the starting weight of said pericarp fraction and adding an acid as needed, and heating to between about 150 to 170° C. for about 11 to 30 minutes;
f. hydrolyzing the pericarp fraction of step (e) by adding enzymes to said pericarp fraction;
g. milling said endosperm and germ fraction to provide a second fines fraction and germ flakes;
h. screening said germ flakes and said second fines fraction to separate said germ flakes and said second fines fraction;
i. combining said first fines fraction and said second fines fraction to provide a combined fines fraction;
j. mixing said combined fines fraction with 2 to 4 times by weight of water to produce a slurry, adjusting pH of the slurry to 5.8, and heating to 87.8° C. for 30 minutes;
k. heating the mixture of step (j) to 110° C. for ten minutes with addition of α-amylase at a dose of 10-20 units/gram of starch;
l. cooling the mixture of step (k) to 70° C., adding α-amylase at a dose of 10-20 units/gram of starch, and holding at 70° C. for 60-120 minutes;
m. combining a hydrolyzed pericarp fraction with the product of step (l) to provide a fermentation media;
n. lowering the temperature of the fermentation media of step (m) to 35° C., adding glucoamylase at a dose of 0.22 units/gram of starch or GLU/gram), and fermenting for 48-64 hours to produce ethanol and stillage.

20. The method of claim 19, further including separating the hydrolyzed pericarp of step (f) into a solid fraction and a liquid stream prior to combining the hydrolyzed pericarp fraction with the fermentation media of step (l), and combining only the liquid stream with the product of step (l).
